(19) United States
(12) Patent Application Publication
(10) Pub. No.: US 2025/0180573 A1
(43) Pub. Date: Jun. 5, 2025

(54) PARTICLE, TEST PARTICLE, REAGENT, AND TEST KIT, AND DETECTION METHOD

(71) Applicant: CANON KABUSHIKI KAISHA, Tokyo (JP)

(72) Inventors: IBUKI ASANO, Kanagawa (JP); SAKAE SUDA, Kanagawa (JP); MASAKI TSUNODA, Tokyo (JP); RYO NATORI, Tokyo (JP); MASASHI KAWAMURA, Kanagawa (JP)

(21) Appl. No.: 18/956,141

(22) Filed: Nov. 22, 2024

(30) Foreign Application Priority Data
Nov. 30, 2023 (JP) 2023-203383

(51) Int. Cl. G01N 33/68; G01N 33/531

(52) U.S. Cl. CPC G01N 33/68; G01N 33/531; G01N 2333/4737

(57) ABSTRACT

Provided are a particle and a test particle improved in redispersibility of the particle, in particular, in redispersibility thereof in a reactive functional group activation step. The particle is a particle including a polymer having a structure represented by the formula (1) and a polymer having a structure represented by the formula (2), wherein a difference between a glass transition point at 90° C. or more and 120° C. or less in a DSC curve obtained at a time of a first temperature increase and a glass transition point at 90° C. or more and 120° C. or less in a DSC curve obtained at a time of a second temperature increase in differential scanning calorimeter (DSC) measurement of the particle has a relationship of −5° C. or more and 5° C. or less.

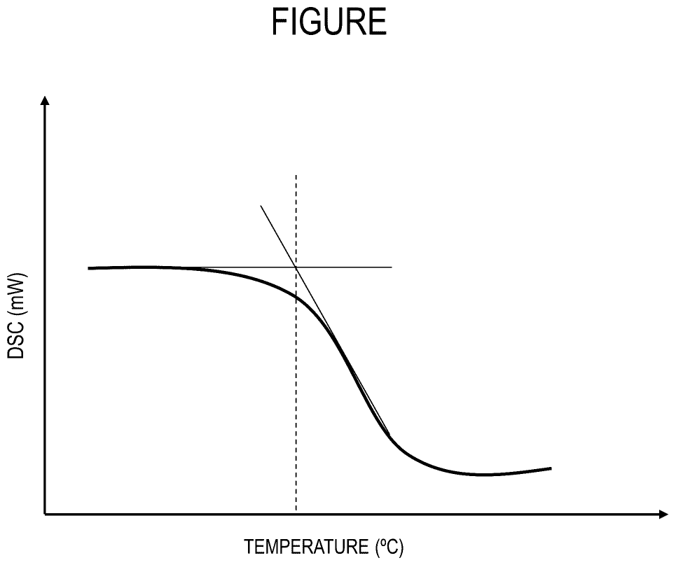

### BACKGROUND OF THE INVENTION

### Field of the Invention

The present invention relates to a particle, a test particle, a reagent, and a test kit, and a detection method.

### Description of the Related Art

In recent years, investigations involving purifying and quantifying a target substance through use of a test particle in which a ligand having an affinity for the target substance is added to a particle surface have been widely made.

In each of Japanese Patent Application Laid-Open No. 2007-224213 and Japanese Patent Application Laid-Open No. 2000-351814, there is a description of a particle having a core-shell structure in which a shell contains, for example, a carboxy group or a 2,3-dihydroxypropyl group, which is a reactive functional group.

Regarding the particle to be used for such purposes, it is preferred that a ligand be uniformly added to a particle surface, to thereby increase an agglutination speed to a target substance and improve sensitivity. When the ligand is added to the particle, the particle is required to be purified by performing sedimentation of the particle with a centrifugal separator or the like, removal of a supernatant, and subsequent redispersion of the particle sediment. In order to uniformly bond the ligand to the particle surface, it is required that the particle be uniformly redispersed in a redispersion step.

In Japanese Patent Application Laid-Open No. 2001-228149, there is a description of a method of controlling surface charge by adding a surfactant or a dispersing aid at the time of preparation of a particle, to thereby enhance redispersibility. However, in the method of controlling surface charge by adding the additive, it becomes difficult to add an antigen (or an antibody) to the particle surface, and the agglutination by an immunoreaction may be inhibited. Accordingly, complicated and cumbersome preparation is required depending on the kind of the antigen (or the antibody) to be bonded. Thus, there is a demand for a method of improving the redispersibility of the particle.

### SUMMARY OF THE INVENTION

An object of the present invention is to provide a particle and a test particle improved in redispersibility of the particle, in particular, in redispersibility thereof in a reactive functional group activation step.

A particle is a particle including a polymer having a structure represented by the formula (1) and a polymer having a structure represented by the formula (2), wherein a difference between a glass transition point at 90° C. or more and 120° C. or less in a DSC curve obtained at a time of a first temperature increase and a glass transition point at 90° C. or more and 120° C. or less in a DSC curve obtained at a time of a second temperature increase in differential scanning calorimeter (DSC) measurement of the particle has a relationship of −5.0° C. or more and 5.0° C. or less:

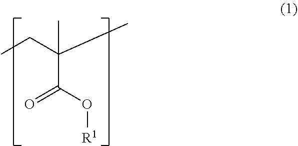

where R¹ represents a group having an epoxy group, a hydroxy group, or a carboxy group, and R¹ may vary depending on each structural unit;

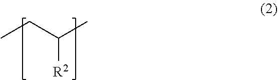

where R² represents a substituted or unsubstituted phenyl group, or a substituted or unsubstituted naphthyl group, provided that a substituent of the substituted group is a methyl group or an ethyl group, and R² may vary depending on each structural unit.

Further features of the present invention will become apparent from the following description of exemplary embodiments with reference to the attached drawing.

### BRIEF DESCRIPTION OF THE DRAWINGS

FIGURE is a graph showing how to determine a grass transition point at the time of a temperature increase in DSC measurement.

### DESCRIPTION OF THE EMBODIMENTS

The present invention is described in detail below by way of exemplary embodiments.

### Problems to be Solved by Particle According to Embodiment of the Present Invention

According to the investigations made by the inventors of the present invention, in the configuration of the particle as described in each of Japanese Patent Application Laid-Open No. 2007-224213 and Japanese Patent Application Laid-Open No. 2000-351814, there have been cases, for example, in which, when the particle is redispersed by centrifugal precipitation treatment, it takes a long time to redisperse the particle, and in which the particle diameter does not become equal to the initial particle diameter after redispersion, and hence there has been a problem in redispersibility. The inventors of the present invention have found that, in particular, when a ligand is chemically bonded to a particle surface, the particles are liable to be agglutinated in a reactive functional group activation step, which makes redispersion further difficult.

In addition, in Japanese Patent Application Laid-Open No. 2001-228149, there is a description of a method involving using an additive in order to improve the redispersibility, but complicated and cumbersome preparation is required in some cases depending on the kind of the ligand to be bonded. The details of the particle are not described, and the reactive functional group activation step is also not described. A particle according to an embodiment of the present invention can solve those problems. The details thereof are described below.

### Particle According to Embodiment of the Present Invention

A particle of the present invention is a particle including a polymer having a structure represented by the following formula (1) and a polymer having a structure represented by the following formula (2), wherein a difference between a glass transition point at 90° C. or more and 120° C. or less in a DSC curve obtained at a time of a first temperature increase and a glass transition point at 90° C. or more and 120° C. or less in a DSC curve obtained at a time of a second temperature increase in differential scanning calorimeter (DSC) measurement of the particle has a relationship of −5.0° C. or more and 5.0° C. or less:

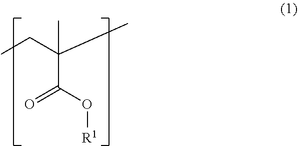

where R¹ represents a group having an epoxy group, a hydroxy group, or a carboxy group, and R¹ may vary depending on each structural unit;

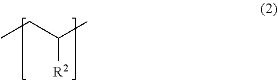

where R² represents a substituted or unsubstituted phenyl group or naphthyl group, provided that a substituent of the substituted group is a methyl group or an ethyl group, and R² may vary depending on each structural unit.

The inventors of the present invention have found that the difference in glass transition point is important in order to obtain a particle having satisfactory redispersibility of the particle, in particular, satisfactory redispersibility thereof in a reactive functional group activation step, and have found that it is particularly important that the difference be −5° C. or more and 5° C. or less.

The particle of the present invention is characterized in that a difference between a glass transition point at 90° C. or more and 120° C. or less in a DSC curve obtained at the time of a first temperature increase and a glass transition point at 90° C. or more and 120° C. or less in a DSC curve obtained at the time of a second temperature increase in differential scanning calorimeter (DSC) measurement is −5.0° C. or more and 5.0° C. or less, and the difference is preferably −4.4° C. or more and 4.4° C. or less, more preferably −2.8° C. or more and 2.8° C. or less.

It is conceived that, when the difference between the glass transition points obtained at the time of the first and second temperature increases in the DSC measurement is smaller, the particle is less liable to be deformed. It is conceived that, when the particle is less liable to be deformed at the time of centrifugation, the contact area between the particles is reduced, and hence the particle is dispersed with a small force, resulting in an improvement in redispersibility of the particle.

It is more preferred that the glass transition points obtained at the time of the first and second temperature increases in the DSC measurement be present at 103° C. or more and 111° C. or less.

The particle of the present invention includes the polymer having the structure represented by the formula (1). In particular, it is preferred that the structure represented by the formula (1) be the structure represented by the following formula (1-A):

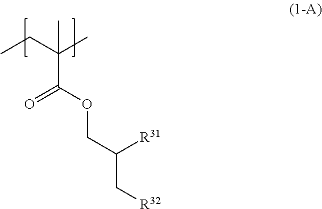

where at least one of R³¹ or R³² represents a hydroxy group, and the other thereof represents a hydroxy group, a group represented by the following formula (1-B), or a group represented by the following formula (1-C);

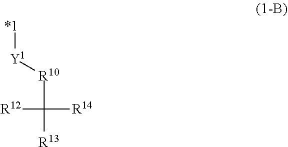

where R¹⁰ represents a single bond or a methylene group, R¹², R¹³, and R¹⁴ each independently represent a hydrogen atom, a methyl group, a hydroxy group, or a hydroxymethyl group, and one or more of R¹², R¹³, and R¹⁴ each represent a hydroxy group, Y¹ represents a sulfur atom or an imino group, and *1 represents a bonding position;

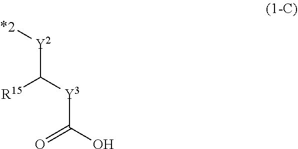

where R¹⁵ represents a hydrogen atom, a methyl group, a hydroxy group, or a carboxy group, Y² represents a sulfur atom or an imino group, Y³ represents a single bond or a methylene group, and *2 represents a bonding position.

Specific structures of the formula (1-A) are shown below but are not limited thereto.

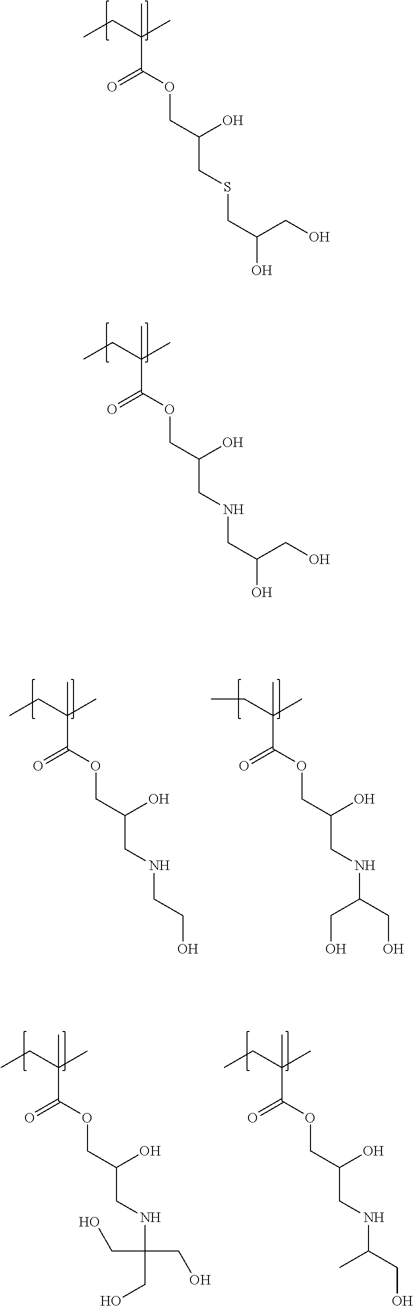

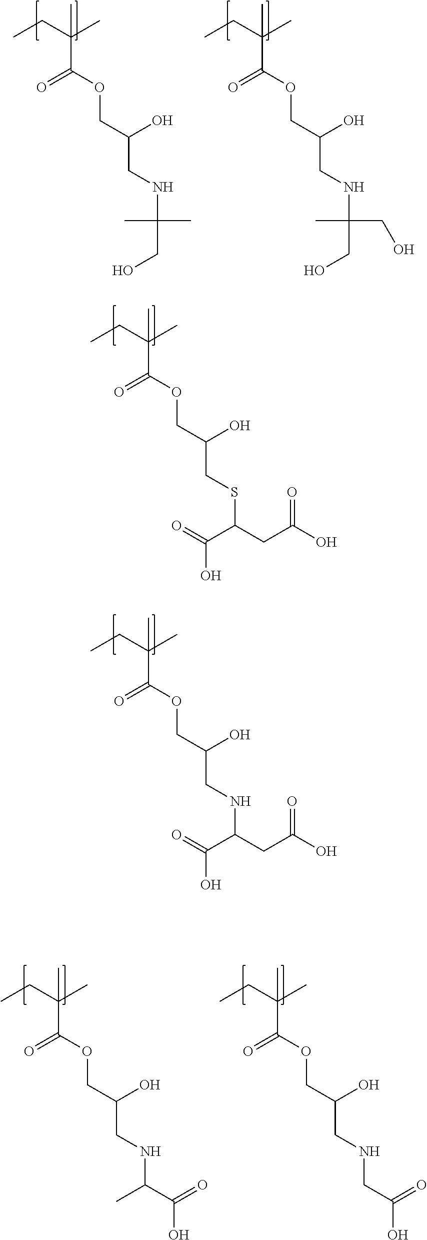

The structure represented by the formula (1-A) of the present invention is obtained by causing a monomer represented by the formula (X2) described later to react with a polymer obtained by polymerizing a monomer represented by the formula (X1) described later. A specific example of the monomer represented by the formula (X1) is not particularly limited, but is preferably glycidyl (meth)acrylate.

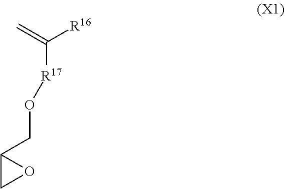

where R¹⁶ represents a hydrogen atom or a methyl group, and R¹⁷ represents an ethylene group or a carbonyl group.

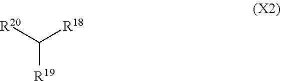

where R¹⁸ represents an amino group or a thiol group, R¹⁹ and R²⁰ each independently represent a hydrogen atom, a methyl group, a group having a hydroxy group, or a group having a carboxy group, and at least one of R¹⁹ or R²⁰ represents a group having a hydroxy group or a group having a carboxy group.

In particular, Y¹ in the formula (1-B) more preferably represents S. When Y¹ represented S in Example described later, the difference between the glass transition points obtained at the time of the first and second temperature increases in the DSC measurement was reduced, and the redispersibility was improved. This is presumably because an S atom has a structure having a higher density in water as compared to a N atom.

The particle of the present invention includes the polymer having the structure represented by the formula (2), and has a substituted or unsubstituted phenyl group or naphthyl group at R². It is known that the phenyl group and the naphthyl group each increase a glass transition point, and the phenyl group and the naphthyl group each increase the grass transition point of the particle by incorporating the repeating unit structure represented by the formula (2) into the particle.

In the particle of the present invention, it is preferred that the molar ratio of the structure represented by the formula (1) with respect to the structure represented by the formula (2) be 0.07 or more and 0.48 or less because the redispersibility is further improved.

At the time of the first temperature increase in the DSC measurement, the endothermic peaks derived from the structure represented by the formula (2) having a high glass transition point and the structure represented by the formula (1) having a low glass transition point appear on a high-temperature side and a low-temperature side, respectively, and hence the structures represented by the formulae (1) and (2) are mixed with each other in the particle at the time of the first temperature increase, resulting in a gradient curve. As a result, it is conceived that, at the time of the second temperature increase in the DSC measurement, the endothermic peak of the structure represented by the formula (2) in the DSC measurement is influenced by the endothermic peak of the structure represented by the formula (1), and there occurs a case in which the endothermic peak may be detected to be lower than that of the first time. Thus, when the molar ratio of the structure represented by the formula (1) with respect to the structure represented by the formula (2) is set to 0.48 or less, there can be provided a particle in which the difference between the glass transition points obtained at the time of the first and second temperature increases in the DSC measurement is small, and which is thus less liable to be deformed, and hence the redispersibility can be improved. In addition, when the molar ratio of the structure represented by the formula (1) with respect to the structure represented by the formula (2) is set to 0.07 or more, the hydrophobicity of the particle surface is reduced, and hence the hydrophobic interaction between the particles is suppressed. Thus, the redispersibility can be improved.

The molar ratio of the structure represented by the formula (1) with respect to the structure represented by the formula (2) may be expressed as a ratio between loaded amounts when the polymerization conversion ratio is recognized to be substantially 100%, but the molar ratio may be measured also by FT-IR from the particle. Specifically, the peak height of a peak that exists in the range of from 1,500 cm⁻¹ to 1,650 cm⁻¹, which is derived from a C═C bond of an aromatic ring, is represented by A, and the peak height of a peak that exists in the range of from 1,680 cm⁻¹ to 1,750 cm⁻¹, which is derived from a carbonyl group of an ester bond, is represented by B, and the value of A/B may be calculated and converted to a molar ratio.

The particle of the present invention preferably has a core-shell structure in which a core is covered with a shell membrane (hereinafter simply referred to as “shell”).

The particle of the present invention more preferably has a core-shell structure in which a core having the structure represented by the formula (2) having a high glass transition point is covered with a shell having the structure represented by the formula (1) having a low glass transition point as described later. The formula (1) has a hydrophilic structure. In addition, it is more preferred that the content ratio of the structure represented by the formula (1) in the shell of the particle be higher than the content ratio of the structure represented by the formula (2) in the core.

When the particle has such core-shell structure, and the content ratio of the structure represented by the formula (1) in the shell of the particle is higher than the content ratio of the structure represented by the formula (2) in the core, the particle is less liable to be deformed at the time of centrifugation, and the hydrophobic interaction can be suppressed, resulting in a further improvement in redispersibility of the particle.

The particle of the present invention may contain a cross-linking agent in the shell. It is conceived that, when the cross-linking agent is incorporated into the shell, the shell has a higher density, and hence the particle is less liable to be deformed at the time of centrifugation, resulting in an improvement in redispersibility of the particle.

The shell of the particle of the present invention has a thickness of preferably 5 nm or more and 35 nm or less, more preferably 10 nm or more and 25 nm or less. When the thickness of the shell is set to 5 nm or more, the hydrophilicity of the particle surface is improved, and hence the hydrophobic interaction between the particles is suppressed, resulting in an improvement in redispersibility of the particle. In addition, when the thickness of the shell is set to 35 nm or less, the particle is less liable to be deformed, and hence the redispersibility is improved.

In addition, the particle of the present invention may have a structure except those represented by the formulae (1) and (2). Examples thereof include structures obtained by polymerizing monomers of styrenes, acrylics, and methacrylics, but the structure is not particularly limited thereto. In addition, the particle may simultaneously have a plurality of structures as the structure except the formulae (1) and (2), but it is more preferred that the weight ratio of the structure represented by the formula (1) in the particle be 9 wt % or more and 39 wt % or less.

The particle of the present invention preferably has a volume-average particle diameter of 150 nm or more and 500 nm or less. When the particle diameter is set to 150 nm or more, there is a tendency that the particle can be easily sedimented by centrifugation. In addition, it is conceived that, when the particle diameter is set to 500 nm or less, the force applied to the particle at the time of centrifugation is suppressed, and hence the particle is less liable to be deformed, resulting in an improvement in redispersibility of the particle.

A value of a ratio (Dv/Dn) between a volume-average particle diameter (Dv) and a number-average particle diameter (Dn) of the particle of present invention is preferably 1.25 or less. Further, the value of Dv/Dn is more preferably less than 1.15. It is known that, as the value of Dv/Dn becomes closer to 1, the particle size distribution is reduced. It is conceived that, when the value of Dv/Dn is set to 1.25 or less, the variation in size of the particles is decreased to reduce the contact area between the particles, to thereby improve the redispersibility.

A method of producing the particle of the present invention is not particularly limited, but soap-free emulsion polymerization is preferred. When the soap-free emulsion polymerization is used, the particle size distribution becomes uniform, and hence the redispersibility is improved. A two-stage swelling polymerization method involving swelling a core with a monomer for forming a shell and forming the shell with a hydrophobic initiator or the like is known as an effective method for uniformizing a particle size distribution, but the core is swollen. It is conceived that, in order to further improve the redispersibility, the reaction is preferably performed under a state of a high particle density in an aqueous solution, and hence it is preferred that the shell be also produced by soap-free emulsion polymerization.

When the particle of the present invention is produced, a polymerization initiator may be used. The polymerization initiator is not particularly limited, but is preferably a hydrophilic polymerization initiator.

A test particle in the present invention has a selectively or specifically high affinity for a target substance by virtue of a ligand added to the particle surface. In particular, the test particle preferably includes a ligand added to the surface of the particle by chemical bonding.

In addition, the ligand in the present invention refers to be a compound that is specifically bonded to a receptor that a specific target substance has. The site at which the ligand is bonded to the target substance is predetermined, and the ligand has a selectively or specifically high affinity for the target substance. Examples of the ligand include: an antigen and an antibody; an enzyme protein and a substrate thereof; a signal substance, such as a hormone or a neurotransmitter, and a receptor thereof; and a nucleic acid. However, the ligand in the present invention is not limited thereto. An example of the nucleic acid is deoxyribonucleic acid. The test particle according to the present invention has a selectively or specifically high affinity for the target substance. The ligand in the present invention is preferably any one of an antibody, an antigen, or a nucleic acid.

A reagent according to the present invention includes the test particle according to the present invention and a dispersion medium that disperses the test particle. The reagent according to the present invention may include a third substance, such as a solvent or a blocking agent, in addition to the test particle according to the present invention to the extent that the object of the present invention can be achieved. The third substance, such as a solvent or a blocking agent, may be incorporated in combination of two or more kinds thereof. Examples of the dispersion medium to be used in the present invention include various buffers, such as a phosphate buffer, a glycine buffer, a Good's buffer, a Tris buffer, and an ammonia buffer, but the dispersion medium contained in the reagent according to the present invention is not limited thereto.

A test kit according to the present invention includes the above-mentioned reagent and a case that encloses the reagent.

The kit according to the present invention may include a reaction buffer (hereinafter referred to as “reagent 2”) in addition to a reagent (hereinafter referred to as “reagent 1”) according to the present invention. A sensitizer may be incorporated into both the reagent 1 and the reagent 2 or any one thereof. In addition, the kit according to the present invention may include a positive control, a negative control, a serum diluent, and the like in addition to the reagent 1 and the reagent 2. In addition to serum or physiological saline free of any target substance that may be measured, a solvent may be used as a medium for the positive control or the negative control.

Although there is no particular limitation on a test method using the particle according to the present invention, a test method using an antibody (antigen) as a ligand and using an antigen (antibody) as a target substance is preferred. Specific examples thereof include: immunochromatography; an immunofluorescence analysis method; chemiluminescence immunoassay; and a latex agglutination method. The particle may be preferably applied to the latex agglutination measurement method, which is widely used in fields, such as clinical testing and biochemical research, out of those methods.

An example of the latex agglutination method in the present invention is a method of detecting a target substance in a specimen, the method including: mixing the test reagent with the specimen that may contain the target substance to provide a mixed liquid; irradiating the mixed liquid with light; and detecting at least any one of transmitted light or scattered light from the light with which the mixed liquid is irradiated.

Examples of a method of redispersing the particle include: a redispersion method by stirring; and a redispersion method by vibration. Examples of the redispersion method by stirring include: a stirring method involving forming a vortex with a vortex mixer or the like; and a method involving directly stirring a precipitate. An example of the redispersion method by vibration is an ultrasonic dispersion method.

The particle according to the present invention may be used as a test particle with respect to a specific target by adding a ligand to the surface of the particle. When the ligand is added to the surface by chemical bonding, it is required to activate the reactive functional groups on the particle surface. This step is referred to as “reactive functional group activation step” in the present invention. A method for the reactive functional group activation step is not particularly limited, and a general method may be used.

When the reactive functional group is a carboxy group, the reactive functional group may be activated through use of a condensing agent and a condensing aid. Examples of the condensing agent include 1-(3-dimethylaminopropyl)-3-ethyl carbodiimide hydrochloride, N,N′-dicyclohexylcarbodiimide, and N,N′-diisopropylcarbodiimide, and 1-(3-dimethylaminopropyl)-3-ethylcarbodiimide hydrochloride is particularly preferred. Examples of the condensing aid include N-hydroxysulfosuccinimide sodium salt, N-hydroxysuccinimide, 1-hydroxybenzotriazole, 1-hydroxy-7-azabenzotriazole, and ethyl cyano(hydroxyimino)acetate, and N-hydroxysulfosuccinimide sodium salt is particularly preferred.

Method of measuring Volume-Average Particle Diameter and Particle Size Distribution of Particle in Aqueous Dispersion

A method of measuring a volume-average particle diameter (Dv) of a particle in an aqueous dispersion in the present invention is described below. The Dv of the particle that exists in an aqueous dispersion in the present invention is measured by a dynamic light scattering method. For example, the Dv is measured at 25° C. with ZETASIZER (ZETASIZER ULTRA: Malvern Panalytical Ltd.). The Dv of a core of the present invention is obtained by measuring an aqueous dispersion of a core obtained in Step-1.In addition, the Dv of a particle including a core and a shell of the present invention is obtained by measuring an aqueous dispersion of a particle obtained in Step-3.

In addition, the particle size distribution of the particle including a core and a shell of the present invention is calculated by measuring the number-average particle diameter (Dn) by the dynamic light scattering method as a ratio (Dv/Dn) of the above-mentioned Dv and Dn.

Method of measuring Thickness of Shell of Particle

A method of calculating the thickness of the shell of the particle in the present invention is described below. The thickness of the shell is calculated by subtracting the volume-average particle diameter of the above-mentioned core from the volume-average particle diameter of the particle obtained by measuring the aqueous dispersion of the particle obtained in Step-3 and dividing the result by 2.

Method of measuring Glass Transition Point of Particle

The glass transition point in the present invention is defined as the temperature at the intersection between a straight line extended from a baseline (a straight line in the range of from 5° C. to 10° C. before the value of the time derivative of pyrolysis is increased) on a low-temperature side to a high-temperature side in the DSC measurement and a tangent line drawn at the point where the slope of a curve in a staircase-like change portion of glass transition becomes maximum (see FIGURE).

The method of measuring the glass transition point of the particle in the present invention is described below. The glass transition point of the particle in the present invention is measured through use of particles weighed in an aluminum pan to be 10 mg or more and 15 mg or less under a state in which the particles are freeze-dried. The measuring device and measuring conditions are as described below.
Measuring device: Differential scanning calorimeter: DSC7020AS-3D (product name, manufactured by Hitachi High-Tech Science Corporation)
Aluminum pan: GAA-0068 (manufactured by Hitachi High-Tech Science Corporation)
Temperature conditions: increasing the temperature from 30° C. to 250° C. at a rate of 10° C./min, decreasing the temperature from 250° C. to 30° C. at a rate of 10° C./min, followed by increasing the temperature again from 30° C. to 250° C. at a rate of 10° C./min.
Analysis software: TA7000 Standard Analysis (manufactured by Hitachi High-Tech Science Corporation)

### Examples

The present invention is described in detail below by way of Examples. However, the present invention is not limited to these Examples.

### [Production Example 1] (Synthesis of Particles 1)

### Step-1

23.52 g of styrene (St: Kishida Chemical Co., Ltd.), 0.43 g of divinylbenzene (DVB: Kishida Chemical Co., Ltd.), and 800.00 g of ion-exchanged water were weighed in a 2 L four-necked separable flask to provide a mixed liquid. After that, the mixed liquid was held at 70° C. while being stirred at 100 rpm, and nitrogen was flowed at a flow rate of 200 ml/min to remove oxygen from the inside of the four-necked separable flask. Next, a separately prepared dissolved liquid, which had been obtained by dissolving 1.02 g of V-50 (FUJIFILM Wako Pure Chemical Corporation) in 30.00 g of ion-exchanged water, was added to the mixed liquid to initiate soap-free emulsion polymerization. The mixed liquid was subjected to a reaction for 48 hours after the initiation of the polymerization to provide a dispersion of particles to be cores (cores 1) each formed of a copolymer of the St and the DVB. Part of the dispersion was collected and evaluated by dynamic light scattering (ZETASIZER ULTRA: Malvern Panalytical Ltd.). As a result, the volume-average particle diameter of the particles was 274 nm.

### Step-2

The solid content concentration of the dispersion obtained in Step-1 was adjusted to 2.0% with ion-exchanged water. The liquid amount of the dispersion after the adjustment was 1,261.52 g. A liquid obtained by collecting and filtering part of this dispersion to remove the particles was evaluated for the contents of the St and the DVB by gas chromatography. As a result, the total content of the St and the DVB was 4 ppm. Next, 12.23 g of glycidyl methacrylate (GMA: Kishida Chemical Co., Ltd.) was added to the liquid. The mixed liquid was decreased in temperature to 70° C. while being stirred at 100 rpm, and nitrogen was flowed at a flow rate of 200 ml/min to remove oxygen from the inside of the four-necked separable flask. Next, a separately prepared dissolved liquid, which had been obtained by dissolving 0.30 g of V-50 in 10.00 g of ion-exchanged water, was added to the mixed liquid to initiate the formation of a shell membrane. The mixed liquid was continuously stirred for 17 hours after the initiation of the reaction to provide a dispersion containing base particles each having a core-shell structure (base particles 1). The dispersion was slowly cooled to room temperature. After that, part of the dispersion was collected, and the polymerization conversion ratio thereof was evaluated by gas chromatography. As a result, it was recognized that the polymerization conversion ratio was substantially 100%.

### Step-3

A previously prepared aqueous solution in which mercaptosuccinic acid (MSA: FUJIFILM Wako Pure Chemical Corporation) and 3-mercapto-1,2-propanediol (MPD: FUJIFILM Wako Pure Chemical Corporation) were dissolved (a content ratio between 3-mercapto-1,2-propanediol and mercaptosuccinic acid was 6:4 (molar fraction), and the total number of moles of the MSA and the MPD was equivalent to the number of moles of the glycidyl methacrylate) was added to the aqueous dispersion containing the base particles 1, and triethylamine (Kishida Chemical Co., Ltd.) was added to adjust the pH to 10. Next, the aqueous dispersion was increased in temperature to 70° C. while being stirred at 200 rpm, and was further held in this state for 18 hours to provide a dispersion of particles 1 each having a core-shell structure. The particles 1 were separated from the dispersion with a centrifugal separator, and the particles 1 were further redispersed in ion-exchanged water; the operation was repeated five times to purify the particles 1, which were stored in the state of an aqueous dispersion in which the concentration of the particles 1 was finally adjusted to 1.0 wt %.

The results obtained by evaluating particle physical properties of the particles 1 and the weight ratio of the formula (1) in the particles 1 are shown in Table 1.

TABLE 1

| | | | | | | Glass |
|---|---|---|---|---|---|---|
| | | | | | | transition point |
| | | | | | | at 90° C. or more |
| | | | | | | and 120° C. or |
| | | | | | | less obtained |
| | | | Volume- | | | at time of first |
| | | | average | | Particle size | temperature |
| | | | particle | Thickness | distribution | increase in |
| | | | diameter Dv of | of shell | of particles | DSC |
| | | | particles | portion | Dv/Dn | measurement |
| | | Particle | (nm) | (nm) | (—) | (° C.) |
| Production | Production Example 1 | Particle 1 | 312 | 19 | 1.14 | 109.8 |
| Example | Production Example 2 | Particle 2 | 286 | 8 | 1.15 | 109.6 |
| | Production Example 3 | Particle 3 | 251 | 16 | 1.14 | 109 |
| | Production Example 4 | Particle 4 | 283 | 7 | 1.13 | 109.3 |
| | Production Example 5 | Particle 5 | 304 | 26 | 1.18 | 106.2 |
| | Production Example 6 | Particle 6 | 315 | 22 | 1.17 | 108.3 |
| Comparative | Comparative Production | Comparative | 250 | — | 1.14 | 116.6 |
| Production | Example 1 | Particle 1 | | | | |
| Example | Comparative Production | Comparative | 2,500 | 760 | 1.15 | 110.9 |
| | Example 2 | Particle 2 | | | | |
| | Comparative Production | Comparative | 301 | 34 | 1.21 | 102.6 |
| | Example 3 | Particle 3 | | | | |

| | | | | Difference | | |
|---|---|---|---|---|---|---|
| | | | | between glass | | |
| | | | Glass | transition | | |
| | | | transition | points | Molar ratio | |
| | | | point at 90° C. | obtained at | of structure | |
| | | | or more and | time of first | represented | |
| | | | 120° C. or less | temperature | by formula | |
| | | | obtained at | increase and | (1) with | |
| | | | time of second | second | respect to | |
| | | | temperature | temperature | structure | |
| | | | increase in | increase in | represented | |
| | | | DSC | DSC | by formula | Weight ratio of |
| | | | measurement | measurement | (2) | formula (1) in |
| | | Particle | (° C.) | (° C.) | (—) | particle (wt %) |
| Production | Production Example 1 | Particle 1 | 110.2 | −0.4 | 0.38 | 34 |
| Example | Production Example 2 | Particle 2 | 106.8 | 2.8 | 0.16 | 18 |
| | Production Example 3 | Particle 3 | 105.6 | 3.4 | 0.38 | 34 |
| | Production Example 4 | Particle 4 | 106.3 | 3 | 0.23 | 24 |
| | Production Example 5 | Particle 5 | 110.6 | −4.4 | 0.38 | 34 |
| | Production Example 6 | Particle 6 | 103.6 | 4.7 | 0.38 | 34 |
| Comparative | Comparative Production | Comparative | 93.2 | 23.4 | 1.3 | 64 |
| Production | Example 1 | Particle 1 | | | | |
| Example | Comparative Production | Comparative | 104 | 6.9 | 1.25 | 63 |
| | Example 2 | Particle 2 | | | | |
| | Comparative Production | Comparative | 107.7 | −5.1 | 0.38 | 34 |
| | Example 3 | Particle 3 | | | | |

### [Production Example 2] (Synthesis of Particles 2)

### Step-1

A dispersion of particles to be cores (cores 2) was produced under the same conditions as in Step-1 of Production Example 1. The volume-average particle diameter of the particles was 270 nm.

### Step-2

A dispersion of base particles (base particles 2) was obtained through use of the dispersion obtained in Step-1 described above by the same experimental operation as in Step-2 of Production Example 1 except that the amount of the GMA was changed from 12.23 g to 5.15 g and the amount of the V-50 was changed from 0.30 g to 0.13 g. The polymerization conversion ratio was evaluated by gas chromatography, and as a result, it was recognized that the polymerization conversion ratio was substantially 100%.

### Step-3

A dispersion of particles 2 each having a core-shell structure was obtained by the same experimental operation as in Step-3 of Production Example 1 except that the base particles 2 were used.

The results obtained by evaluating particle physical properties of the particles 2 and the weight ratio of the formula (1) in the particles 2 are shown in Table 1.

### [Production Example 3] (Synthesis of Particles 3)

### Step-1

25.35 g of styrene (St: Kishida Chemical Co., Ltd.), 0.46 g of divinylbenzene (DVB: Kishida Chemical Co., Ltd.), and 1,491.02 g of ion-exchanged water were weighed in a 2 L four-necked separable flask to provide a mixed liquid. After that, the mixed liquid was held at 70° C. while being stirred at 140 rpm, and nitrogen was flowed at a flow rate of 200 ml/min to remove oxygen from the inside of the four-necked separable flask. Next, a separately prepared dissolved liquid, which had been obtained by dissolving 1.10 g of V-50 (FUJIFILM Wako Pure Chemical Corporation) in 30.00 g of ion-exchanged water, was added to the mixed liquid to initiate soap-free emulsion polymerization. The mixed liquid was subjected to a reaction for 48 hours after the initiation of the polymerization to provide a dispersion of particles (cores 3) each formed of a copolymer of the St and the DVB. Part of the dispersion was collected, and the volume-average particle diameter of the cores 3 was evaluated by dynamic light scattering (ZETASIZER ULTRA: Malvern Panalytical Ltd.). As a result, the volume-average particle diameter was 219 nm.

### Step-2

A dispersion of base particles 3 was obtained through use of the dispersion obtained in Step-1 described above by the same experimental operation as in Step-2 of Production Example 1 except that the amount of the GMA was changed from 12.23 g to 13.18 g and the amount of the V-50 was changed from 0.30 g to 0.32 g. The polymerization conversion ratio was evaluated by gas chromatography, and as a result, it was recognized that the polymerization conversion ratio was substantially 100%

### Step-3

A dispersion of particles 3 each having a core-shell structure was obtained by the same experimental operation as in Step-3 of Production Example 1 except that the base particles 3 were used.

The results obtained by evaluating particle physical properties of the particles 3 and the weight ratio of the formula (1) in the particles 3 are shown in Table 1.

### [Production Example 4] (Synthesis of Particles 4)

### Step-1

A dispersion of particles to be cores (cores 4) was produced under the same conditions as in Step-1 of Production Example 1. The volume-average particle diameter was 270 nm.

### Step-2

A dispersion of base particles 4 was obtained through use of the dispersion obtained in Step-1 described above by the same experimental operation as in Step-2 of Production Example 1 except that the amount of the GMA was changed from 12.23 g to 7.51 g and the amount of the V-50 was changed from 0.30 g to 0.18 g. The polymerization conversion ratio was evaluated by gas chromatography, and as a result, it was recognized that the polymerization conversion ratio was substantially 100%

### Step-3

A dispersion of particles 4 each having a core-shell structure was obtained by the same experimental operation as in Step-3 of Production Example 1 except that the base particles 4 were used.

The results obtained by evaluating particle physical properties of the particles 4 and the weight ratio of the formula (1) in the particles 4 are shown in Table 1.

### [Production Example 5] (Synthesis of Particles 5)

### Step-1

A dispersion of particles (cores 5) was obtained by the same experimental operation as in Step-1 of Production Example 1 except that the amount of the DVB to be used was changed from 0.43 g to 1.47 g. Part of the dispersion was collected and evaluated by dynamic light scattering (ZETASIZER ULTRA: Malvern Panalytical Ltd.). As a result, the volume-average particle diameter was 253 nm.

### Step-2

A dispersion of base particles 5 was obtained through use of the dispersion of cores 5 by the same experimental operation as in Step-2 of Production Example 1 except that the DVB was added in an amount of 1.16 g simultaneously with the GMA and the amount of the V-50 was changed from 0.30 g to 0.13 g. The polymerization conversion ratio was evaluated by gas chromatography, and as a result, it was recognized that the polymerization conversion ratio was substantially 100%

### Step-3

A dispersion of particles 5 each having a core-shell structure was obtained by the same experimental operation as in Step-3 of Production Example 1 except that the base particles 5 were used.

The results obtained by evaluating particle physical properties of the particles 5 and the weight ratio of the formula (1) in the particles 5 are shown in Table 1.

### [Production Example 6] (Synthesis of Particles 6)

### Step-1

A dispersion of particles to be cores (cores 6) was produced under the same conditions as in Step-1 of Production Example 1. The volume-average particle diameter was 271 nm.

### Step-2

A dispersion of base particles 6 was obtained by the same experimental operation as in Step-2 of Production Example 1.

### Step-3

A dispersion of particles 6 each having a core-shell structure was obtained by the same experimental operation as in Step-3 of Production Example 1 except that: the MPD was changed to an aqueous solution obtained by dissolving 2-amino-2-hydroxymethyl-1,3-propanediol hydrochloride (Tris: Tokyo Chemical Industry Co., Ltd.); and the total number of moles of the MSA and the Tris was changed from the number of moles equivalent to that of the glycidyl methacrylate to the number of moles that was twice as large as that of the glycidyl methacrylate.

The results obtained by evaluating particle physical properties of the particles 6 and the weight ratio of the formula (1) in the particles 6 are shown in Table 1.

### [Comparative Production Example 1] (Synthesis of Comparative Particles 1)

The St, the GMA, the DVB, the V-50, and ion-exchanged water were mixed in accordance with the following formulation: St/GMA/DVB/V-50/H₂O=1.2/1.8+0.3/0.04/0.06/110 (g). After nitrogen purging, a polymerization reaction was performed at 70° C. for 24 hours. Two hours after the initiation of the polymerization, 0.3 g of the GMA was added to the mixture to provide a dispersion of comparative base particles 1. The resultant comparative base particles 1 were sedimented by centrifugation (15,000 rpm, 15 min, 4° C.), and the supernatant was subjected to decantation. Then, the comparative base particles 1 were redispersed in 200 ml of water. The comparative base particles 1 were washed by repeating the above-mentioned operation three times, and were finally dispersed in water.

In order to introduce amino groups into the washed base particles 1 (0.25 g), NH₄OH (55.3 mmol; corresponding to 50 times as large as the amount of a GMA unit) was added, and the dispersion was adjusted to pH 11 with 1 N HCl. The dispersion was subjected to a reaction at 70° C. for 24 hours while being stirred with a stirrer so that the epoxy groups of the GMA were opened. Next, an example in which ethylene glycol diglycidyl ether (EGDE; Wako Pure Chemical Industries, Ltd.) was immobilized on the comparative base particles 1 obtained above is described below. An excess amount of the EGDE was loaded so that the amount thereof was 100 times (mol) as large as that of the amino groups of the comparative base particles 2 in an amount of about 62.5 mg, followed by stirring at 30° C. for 24 hours at pH 11 (adjusted with 1 N NaOH), to thereby covalently bond the epoxy groups of the EGDE to the amino groups on the comparative base particles 1. An excess amount of the EGDE was added in order to prevent the epoxy groups present on both terminals of one of the EGDE1 molecules from being simultaneously immobilized on the comparative base particles 1. As a result, a dispersion of particles (EGDE particles) in which the EGDE was bonded to the comparative base particles 1 was obtained. After the reaction, the particles were washed three times with water by a centrifugal operation.

In order to introduce amino groups into 0.25 g of the washed EGDE particles, NH₄OH (55.3 mmol; corresponding to 50 times as large as the amount of a GMA unit) was added, and the dispersion was adjusted to pH 11 with 1 N HCl. The dispersion was subjected to a reaction at 70° C. for 24 hours while being stirred with a stirrer. Then, the dispersion was purified by centrifugation through use of ion-exchanged water three times under the conditions of 4° C. and 27,000 G for 20 minutes, and then the particles were redispersed in methanol so that the solid content ratio was 1 wt %. Next, 0.88 g of succinic anhydride (Tokyo Chemical Industry Co., Ltd.) was added to a dispersion 1 that was weighed so that the amount of the particles was 0.20 g. The mixture was shaken at 30° C. for 5 hours to cause primary amines of the particles having the amino groups introduced into the EGDE terminals to react with the succinic anhydride to provide comparative particles 1.

The results obtained by evaluating particle physical properties of the comparative particles 1 are shown in Table 1.

### [Comparative Production Example 2] (Synthesis of Comparative Particles 2)

Cores produced under the same conditions as in Step-1 of Production Example 1 were prepared in 500 g of water that the solid content thereof was 5.0 g. An organic solvent (Shellsol TK: manufactured by Shell Chemicals Japan Ltd., 0.1 g) and 4.66 g of the GMA were added thereto in the stated order, followed by stirring. After that, 2 g of azobisisobutyronitrile (AIBN) was added to the mixture, followed by slow stirring at 75° C. for 24 hours, to form a polymer portion. Next, the reaction liquid was cooled and then filtered through a 500-mesh wire mesh. The particles were washed with distilled water by centrifugation to provide a dispersion of comparative particles 2 each having a core-shell structure.

The results obtained by evaluating particle physical properties of the comparative particles 2 are shown in Table 1.

### [Comparative Production Example 3] (Synthesis of Comparative Particles 3)

### Step-1

A dispersion of particles (comparative core particles 3) was obtained by the same experimental operation as in Step-1 of Production Example 1 except that the polymerization time was changed from 48 hours to 3 hours. Part of the dispersion was collected and evaluated by dynamic light scattering (ZETASIZER ULTRA: Malvern Panalytical Ltd.). As a result, the volume-average particle diameter was 233.5 nm.

### Step-2

A dispersion of comparative base particles 3 was obtained by the same experimental operation as in Step-2 of Production Example 1.

### Step-3

A dispersion of comparative particles 3 each having a core-shell structure was obtained by the same experimental operation as in Step-3 of Production Example 1 except that the comparative base particles 3 were used.

The results obtained by evaluating particle physical properties of the comparative particles 3 and the weight ratio of the formula (1) in the comparative particles 3 are shown in Table 1.

[Evaluation 1] Evaluation of Redispersibility of Particle having Activated Carboxy Group (Particle Diameter Basis)

### Reactive Functional Group Activation Step of Particle

180 μL of a 1.7 wt % water suspension of the particles produced in each of Production Examples 1 to 6 and Comparative Production Examples 1 to 3 was dispensed in a 1.5 mL microtube. Then, 90 μL of a 5.0% aqueous solution of 1-(3-dimethylaminopropyl)-3-ethylcarbodiimide hydrochloride and 90 μL of a 5.0% aqueous solution of N-hydroxysulfosuccinimide sodium salt were added as condensing agents to the suspension, followed by stirring at room temperature for 30 minutes, to provide particles each having an activated carboxy group (hereinafter referred to as “activated particles”).

### Redispersion Step

A dispersion of the activated particles produced above was sedimented at a force of 20,000 G for 5 minutes with a centrifugal separator. The supernatant was drained, and 250 μL of a Tris-HCl buffer having a pH of 8.0 was newly added. Six microtubes each containing the activated particle sediment having the buffer added thereto were prepared, and ultrasonic dispersion treatment with an ultrasonic disintegrator Bioruptor II (TYPE 6) manufactured by Sonicbio Co., Ltd. in a High mode at a liquid temperature of 4° C. for 16 seconds was performed 15 times.

### Evaluation of Redispersibility

The state of each of the particles in the microtubes after the ultrasonic dispersion treatment was visually checked. After that, the particle diameter of each of the particles was measured with ZETASIZER ULTRA manufactured by Spectris Co., Ltd., and an average value of changes in particle diameter before and after the activation in the six microtubes was calculated. The resultant average value was evaluated as described below.
A: (Particle diameter of activated particles after ultrasonic dispersion)/(particle diameter of particles before activation)=1.05 or less, and a uniform solution free of any precipitate was visually observed when the ultrasonic dispersion treatment for 16 seconds was performed six times.
B: (Particle diameter of activated particles after ultrasonic dispersion)/(particle diameter of particles before activation)=1.05 or less
C: (Particle diameter of activated particles after ultrasonic dispersion)/(particle diameter of particles before activation)=more than 1.05 and 1.1 or less
D: (Particle diameter of activated particles after ultrasonic dispersion)/(particle diameter of particles before activation)=more than 1.1 and 2.0 or less
E: A remaining sediment was visually observed.

The evaluation results of the activated particles of the particles 1 to 6 and the comparative particles 1 to 3 are shown in Table 2. Regarding all the particles 1 to 6, the evaluation results were C or more.

Meanwhile, regarding all the comparative particles 1 to 3, the evaluation results were E. Regarding the comparative particles 1 and 2, the weight ratios of the formula (1) in the particles were 64% and 63%, respectively, as shown in Table 1, indicating that the ratio of the structure represented by the formula (1) having a low glass transition point was high, and hence the comparative particles 1 and 2 were soft and liable to be deformed as compared to the particles 1 to 6. As a result, the redispersibility is decreased. Regarding the comparative particles 3, the polymerization time is short. Because of this, the cross-linking of the core particles is insufficient, and St remains in the solution. Thus, the glass transition point at the time of the first temperature increase is lower than the glass transition point at the time of the second temperature increase as shown in Table 1, and hence the comparative particles 3 are soft and liable to be deformed, with the result that the redispersibility is decreased.

[Evaluation 2] Evaluation of Redispersibility of Particle having Activated Carboxy Group (Time Basis)

### Reactive Functional Group Activation Step of Particle

180 μL of a 1.7 wt % water suspension of the particles produced in each of Production Examples 1 to 6 and Comparative Production Examples 1 to 3 was dispensed in a 1.5 mL microtube. Then, 90 μL of a 5.0% aqueous solution of 1-(3-dimethylaminopropyl)-3-ethylcarbodiimide hydrochloride and 90 μL of a 5.0% aqueous solution of N-hydroxysulfosuccinimide sodium salt were added as condensing agents to the suspension, followed by stirring at room temperature for 30 minutes, to provide activated particles.

### Redispersion Step

A dispersion of the activated particles produced above was sedimented at a force of 20,000 G for 5 minutes with a centrifugal separator. The supernatant was drained, and 250 μL of a Tris-HCl buffer having a pH of 8.0 was newly added. Six microtubes each containing the activated particle sediment having the buffer added thereto were prepared, and ultrasonic dispersion treatment was performed with an ultrasonic disintegrator Bioruptor II (TYPE 6) manufactured by Sonicbio Co., Ltd. in a High mode at a liquid temperature of 4° C. for 16 seconds. The state of each of the particles in the microtubes after the ultrasonic dispersion treatment was visually observed for checking whether or not the solution in which the particles were dispersed without any precipitate was uniformly cloudy. When there was a precipitate or the cloudiness of the solution in which the particles were dispersed was non-uniform, the ultrasonic dispersion treatment was performed for an additional 16 seconds, and visual observation was performed. This operation was repeated until the solution in which the particles were dispersed without any precipitate became uniformly cloudy.

### Evaluation of Redispersibility

An average value of the times taken for visually recognizing that the solution in which the particles were dispersed without any precipitate became uniformly cloudy in the six microtubes was calculated and evaluated as described below.
A: 30 seconds or less
B: 45 seconds or less
C: 60 seconds or less
D: 61 seconds or more

The evaluation results of the activated particles of the particles 1 to 6 and the comparative particles 1 to 3 are shown in Table 2. Regarding all the particles 1 to 6, the evaluation results were C or more. When the average redispersion time of the particle is short, and the redispersibility is high, damage to a ligand becomes small when the particle having the ligand added thereto is redispersed by ultrasonic irradiation. When the damage to the ligand is small, the reactivity becomes high when the particle becomes a test particle, and hence the sensitivity becomes high.

Meanwhile, regarding all the comparative particles 1 to 3, the evaluation results were D. Regarding the comparative particles 1 and 2, the weight ratios of the formula (1) in the particles were 64% and 63%, respectively, as shown in Table 1, indicating that the ratio of the structure represented by the formula (1) having a low glass transition point was high, and hence the comparative particles 1 and 2 were soft and liable to be deformed as compared to the particles 1 to 6. As a result, the redispersibility is decreased. Regarding the comparative particles 3, the polymerization time is short. Because of this, the cross-linking of the core particles is insufficient, and St remains in the solution. Thus, the glass transition point at the time of the first temperature increase is lower than the glass transition point at the time of the second temperature increase as shown in Table 1, and the comparative particles 3 are soft and liable to be deformed, with the result that the redispersibility is decreased.

[Evaluation 3] Evaluation of Nonspecific Adsorptivity with Respect to Particle

### Evaluation of Chyle

The particles 1 were dispersed in a phosphate buffer at 0.1 wt % to prepare a dispersion. Next, 60 μL of a chyle liquid formed of triolein, lecithin, free fatty acids, bovine albumin, and a Tris buffer was added to 30 μL of the dispersion, and the absorbance of the dispersion immediately after its stirring at a wavelength of 572 nm was measured. A spectrophotometer Biospectrophotometer manufactured by Eppendorf SE was used in the absorbance measurement. Then, the dispersion was left at rest at 37° C. for 5 minutes, and then its absorbance at a wavelength of 572 nm was measured again, followed by the calculation of a value of “(variation ΔABS in absorbance)×10,000.” The evaluation was performed based on the value of ΔABS×10,000 as described below.
A: ΔABS×10,000 of 30 or less
B: ΔABS×10,000 of more than 30 and 50 or less
C: ΔABS×10,000 of more than 50 and 100 or less
D: ΔABS×10,000 of more than 100 and 500 or less
E: ΔABS×10,000 of 500 or more

The evaluation result was A. It was recognized that the particle according to the present invention was excellent in ability to suppress nonspecific adsorption.

### [Evaluation 4] Production of Test Particle by Antibody Sensitization of Particle and Evaluation of Latex Agglutination Sensitivity of Test Particle

### Production of Test Particle by Antibody Sensitization of Particle

180 μL of a 1.7 wt % water suspension of the particles 1 was dispensed in a 1.5 mL microtube, and 90 μL of a 5.0% aqueous solution of 1-(3-dimethylaminopropyl)-3-ethylcarbodiimide hydrochloride and 90 μL of a 5.0% aqueous solution of N-hydroxysulfosuccinimide sodium salt were added thereto. The mixture was stirred at room temperature for 30 minutes to provide a dispersion of particles each having an activated carboxy group (activated particle dispersion).

After centrifugal washing, 270 μL of phosphate buffered saline (hereinafter referred to as “PBS”) having a pH of 7.2 was added to the resultant, and the particles each having an activated carboxy group were dispersed with an ultrasonic wave.

5 μL of a 15.0 mg/mL dispersion of clone C5 (Funakoshi Co., Ltd.) of a monoclonal mouse anti-human C-reactive protein (hereinafter referred to as “CRP antibody”) was added thereto. The mixture was stirred at room temperature for 3 hours to provide test particles by antibody sensitization of the particles. Those test particles were subjected to centrifugal washing. After that, 1 mL of the PBS was added to the resultant, and the test particles were stored in a dispersed state.

### Evaluation of Latex Agglutination Sensitivity of Test Particle

Standard serum for CRP was diluted with the PBS to a concentration of 0.75 mg/dL, and the resultant was used as a CRP specimen solution. A mixed liquid obtained by mixing 1 μL of the CRP specimen solution and 50 μL of a buffer (PBS containing 0.01% Tween 20) (hereinafter referred to as “R1”) was prepared and held at 37° C.

Next, 50 μL of each of the above-mentioned dispersions of affinity particles in which the affinity particles were sufficiently dispersed again with an ultrasonic wave before use (particle concentration: 0.1 wt %, referred to as “R2”) was mixed with the R1. The absorbance at a wavelength of 572 nm of the mixed liquid (volume: 101 μL) immediately after stirring was measured. A spectrophotometer Biospectrophotometer manufactured by Eppendorf SE was used in the absorbance measurement. Then, the mixed liquid was left at rest at 37° C. for 5 minutes, and then its absorbance at a wavelength of 572 nm was measured again, followed by the calculation of the value of “(variation ΔABS in absorbance)×10,000.”

The value was calculated to be 10,000 or more, which was satisfactory as an evaluation result. It was recognized that the particle according to the present invention had a sufficient ability as a test particle. Regarding the particles 2 to 6, satisfactory results are expected also in Evaluations 3 and 4.

TABLE 2

| | | | [Evaluation 1] | [Evaluation 2] | |
|---|---|---|---|---|---|
| | | | Evaluation of | Evaluation of redispersibility of | |
| | | | redispersibility of | activated particle | |
| | | | activated particle | | Average |
| | | | Particle diameter | Time | redispersion |
| | | Particle | basis | basis | time (sec) |
| Example | Example 1 | Particle 1 | A | A | 27 |
| | Example 2 | Particle 2 | A | B | 40 |
| | Example 3 | Particle 3 | B | B | 37 |
| | Example 4 | Particle 4 | B | B | 37 |
| | Example 5 | Particle 5 | B | C | 53 |
| | Example 6 | Particle 6 | C | A | 29 |
| Comparative | Comparative | Comparative | E | D | 216 |
| Example | Example 1 | Particle 1 | | | |
| | Comparative | Comparative | E | D | 80 |
| | Example 2 | Particle 2 | | | |
| | Comparative | Comparative | E | D | 112 |
| | Example 3 | Particle 3 | | | |

As described above, the particles 1 to 6 of the present invention have high redispersibility in Evaluation 1. Meanwhile, the comparative particles 1 to 3 have low redispersibility.

According to the present invention, there are provided a particle and a test particle improved in redispersibility of the particle, in particular, in redispersibility thereof in the reactive functional group activation step.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-203383, filed Nov. 30, 2023, which is hereby incorporated by reference herein in its entirety.

1. A particle comprising a polymer having a structure represented by formula (1) and a polymer having a structure represented by formula (2),
wherein a difference between a glass transition point at 90° C. to 120° C. in a DSC curve obtained at a time of a first temperature increase and a glass transition point at 90° C. to 120° C. in a DSC curve obtained at a time of a second temperature increase in differential scanning calorimeter (DSC) measurement of the particle is −5.0° C. to 5.0° C.: 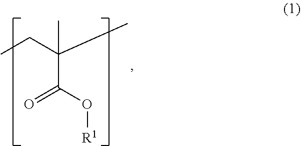
where R¹ represents a group having an epoxy group, a hydroxy group, or a carboxy group, and R¹ may vary depending on each structural unit; 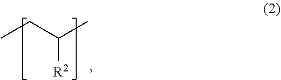
where R² represents a substituted or unsubstituted phenyl group, or a substituted or unsubstituted naphthyl group, provided that a substituent of the substituted group is a methyl group or an ethyl group, and R² may vary depending on each structural unit.

2. The particle according to claim 1, wherein a molar ratio of the structure represented by the formula (1) with respect to the structure represented by the formula (2) is 0.07 to 0.48.

3. The particle according to claim 1, wherein the particle has a core-shell structure including a core and a shell.

4. The particle according to claim 3, wherein the shell contains the polymer having the structure represented by the formula (1), and the core contains the polymer having the structure represented by the formula (2).

5. The particle according to claim 4, wherein a content ratio of the structure represented by the formula (1) in the shell is higher than a content ratio of the structure represented by the formula (2) in the core.

6. The particle according to claim 3, wherein the shell has a thickness of 5 nm to 35 nm.

7. The particle according to claim 1, wherein the difference between the glass transition point at 90° C. to 120° C. in the DSC curve obtained at the time of the first temperature increase and the glass transition point at 90° C. to 120° C. in the DSC curve obtained at the time of the second temperature increase in the differential scanning calorimeter (DSC) measurement is −4.4° C. to 4.4° C.

8. The particle according to claim 7, wherein the difference between the glass transition point at 90° C. to 120° C. in the DSC curve obtained at the time of the first temperature increase and the glass transition point at 90° C. to 120° C. in the DSC curve obtained at the time of the second temperature increase in the differential scanning calorimeter (DSC) measurement is −2.8° C. to 2.8° C.

9. The particle according to claim 1, wherein the particle has a volume-average particle diameter of 150 nm to 500 nm.

10. The particle according to claim 1, wherein a content of the structure represented by the formula (1) in the particle is 9 wt % to 39 wt %.

11. The particle according to claim 1, wherein the formula (1) is represented by formula (1-A): 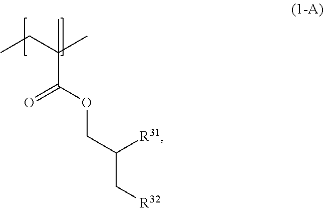
where at least one of R³¹ or R³² represents a hydroxy group, and another thereof represents a hydroxy group, a group represented by formula (1-B), or a group represented by formula (1-C): 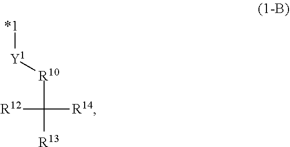
where R¹⁰ represents a single bond or a methylene group, R¹², R¹³, and R¹⁴ each independently represent a hydrogen atom, a methyl group, a hydroxy group, or a hydroxymethyl group, and one or more of R¹², R¹³, and R¹⁴ each represent a hydroxy group, Y¹ represents a sulfur atom or an imino group, and *1 represents a bonding position; 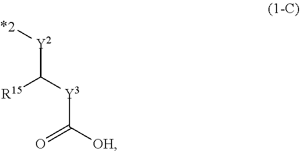
where R¹⁵ represents a hydrogen atom, a methyl group, a hydroxy group, or a carboxy group, Y² represents a sulfur atom or an imino group, Y³ represents a single bond or a methylene group, and *2 represents a bonding position.

12. The particle according to claim 11, wherein Y¹ in the formula (1-B) represents a sulfur atom.

13. A test particle comprising a ligand added to a surface of the particle of claim 1.

14. A reagent comprising the test particle of claim 13 dispersed in an aqueous solution.

15. A test kit comprising the reagent of claim 14 and a case configured to enclose the reagent.

16. A method of detecting a target substance in a specimen, the method comprising mixing the reagent of claim 14 and the specimen that may contain the target substance.

17. A method of detecting a target substance in a specimen by a latex agglutination method, the method comprising:
mixing the reagent of claim 14 with the specimen that may contain the target substance to provide a mixed liquid;
irradiating the mixed liquid with light; and
detecting at least any one of transmitted light or scattered light from the light with which the mixed liquid is irradiated.
